(19) United States
(12) Patent Application Publication
(10) Pub. No.: US 2023/0002715 A1
(43) Pub. Date: Jan. 5, 2023

(54) MODULAR PROCESSING SYSTEM

(71) Applicant: Sartorius Stedim Biotech GmbH, Göttingen (DE)

(72) Inventors: Lars Boettcher, Melsungen (DE); Christian Manzke, Bovenden (DE)

(73) Assignee: Sartorius Stedim Biotech GmbH, Göttingen (DE)

(21) Appl. No.: 17/783,785

(22) PCT Filed: Dec. 9, 2020
(86) PCT No.: PCT/EP2020/085256

§ 371 Date: Jun. 9, 2022

(30) Foreign Application Priority Data
Dec. 9, 2019 (EP) 19214525.8

(51) Int. Cl. C12M 1/00; C12M 3/00; C12M 1/02; C12M 1/34

(52) U.S. Cl. CPC C12M 23/28; C12M 23/42; C12M 23/44; C12M 27/00; C12M 41/26; C12M 41/40; C12M 41/44

(57) ABSTRACT

The invention relates to a modular processing system for biopharmaceutical and/or chemical processes, comprising: at least one processing unit; at least one adapter plate, which can be directly or indirectly fluidically connected to the processing unit, wherein the adapter plate has at least one adapter channel, through which at least one fluid flow can flow to the processing unit, wherein the adapter plate also has at least one deflection element and/or a pump and/or at least one valve; and an external control device. The adapter plate is designed in such a way that the fluid flow to the processing unit can be at least partially deflected with the at least one deflection element in the adapter channel and/or the fluid flow, preferably its pressure, is controllable with the at least one valve and/or the pump in the adapter channel. A respective at least one sensor is embedded in the processing unit and/or in the adapter plate, in order to detect at least one property of the fluid flow in the processing unit or the adapter plate. The external control device can be coupled to the at least one sensor in such a way that measurement data of at least one sensor can be read out, and the fluid flow in the processing unit and or the adapter plate can be centrally controlled based on the read-out measurement data. The invention also relates to a method for centrally controlling a modular processing system for biopharmaceutical and/or chemical processes.

### TECHNICAL FIELD

The present invention relates to a modular processing system for biopharmaceutical and/or chemical processes and to a method for centrally controlling a modular processing system for biopharmaceutical and/or chemical processes.

### BACKGROUND

Processes such as cell separation (for example by depth filtration), sterile filtration, chromatography steps, virus inactivation, virus filtration, and or crossflow filtration are known from the biopharmaceutical sector. All these processes are basic operations that are regularly employed in various overall processes. Processing systems that employ such overall processes require a comprehensive monitoring of the fluid flow or the fluid to ensure that the fluid has the required parameters in the overall process. However, this requires a plurality of measurement points in the processing system and elaborate management of the comprehensive monitoring data.

### SUMMARY

It is then the task of the invention to provide a processing system for biopharmaceutical and/or chemical processes that simplifies monitoring the fluid in the processing system.

This task is solved by a modular processing system for biopharmaceutical and/or chemical processes, comprising:

at least one processing unit;

at least one adapter plate that can be directly or indirectly fluidically connected to the processing unit, wherein the adapter plate has at least one adapter channel through which at least one fluid flow can flow, which flows to the processing unit, wherein the adapter plate additionally has at least one deflection element and/or a pump and/or at least one valve; and

an external control device;

wherein the adapter plate is designed in such a way that the fluid flow to the processing unit can be at least partially deflected with the at least one deflection element in the adapter channel and/or the fluid flow, preferably its pressure, is controllable with the at least one valve and/or the pump in the adapter channel;

wherein at least one sensor is embedded in the processing unit and/or in the adapter plate, in order to detect at least one property of the fluid flow in the processing unit or the adapter plate; and

wherein the external control device can be coupled (directly or indirectly) to the at least one sensor in such a way that measurement data of at least one sensor can be read out, and the fluid flow in the processing unit and or the adapter plate can be centrally controlled based on the read-out measurement data.

A “processing unit” is in particular defined as a unit within which a special processing step for the desired method is performed. A processing unit in particular separates components of a fluid flow. A processing unit can for example be a unit for cell separation (for example by depth filtration), for sterile filtration, for a chromatography step, for virus inactivation, or for crossflow filtration.

In relation to the fluid flow, the adapter plate is arranged upstream of the processing unit such that the fluid flow flows from the adapter plate to the processing unit.

The adapter plate, which can be arranged upstream of a processing unit in a modular processing system, permits making required adjustments concerning the fluid flow. This involves at least partially redirecting or deflecting the fluid flow, or changing the direction of flow of the fluid flow, and/or the fluid flow can be regulated or controlled with the adapter plate, preferably or in particular its pressure, with which the fluid reaches the processing unit.

However, both adjustments can also be made simultaneously within one adapter plate. A deflection can preferably be achieved with at least one deflection element that is located in or on the adapter channel. Additionally or alternatively, a pump and/or at least one valve can be located in the adapter channel, by which the pressure of the fluid flow in the adapter channel can be regulated or controlled.

An adapter plate can then be arranged upstream of or on a processing unit in a processing system to make the required adjustments concerning the fluid flow.

Arranging an adapter plate upstream of a processing unit in a modular processing system provides the ability to readily feed a fluid flow to the downstream processing unit in the manner required for the subsequent processing step in the processing system. Based on the construction, the adapter plate, in combination with a configuration of a processing unit, provides a compact construction such that the required footprint and system components can be reduced. The required investments are reduced accordingly.

Furthermore, at least one sensor that can detect at least one property of the fluid flow is embedded in the processing system in the processing unit and/or in the adapter plate. As a result, measurements can be taken on the fluid flow at preferred positions, thus allowing the fluid flow to be permanently monitored. By embedding the at least one sensor in the processing unit and/or adapter plate, it is in particular not necessary to separately connect a sensor. The latter is already embedded or specified by the factory into the adapter plate or the processing unit, and can be advantageously preinstalled and/or sterilized. The user can as a result skip the work steps of calibrating or sterilizing.

The measurement data of the at least one sensor in the processing unit and/or the adapter plate can be read by an external control device.

If the measurement data deviate from predefined optimum values, the external control device can centrally regulate the fluid flow in the adapter channel and/or the processing unit.

The modular processing system then only requires an external regulating or control device to obtain measurement data from the fluid flow and to regulate the fluid flow in the processing system.

The processing system preferably comprises at least a first and a second processing unit that can be fluidically connected to each other,

wherein at least one fluid flow that flows from the first processing unit to the second processing unit can flow through the at least one adapter channel of the adapter plate; and

wherein the adapter plate is designed in such a way that the fluid flow between the first processing unit and the second the processing unit can be at least partially deflected with the at least one deflection element in the adapter channel and the fluid flow, preferably its pressure, is controllable with the at least one valve and/or the pump in the adapter channel.

The first and second processing unit can have an identical design or have at least partially different properties. The first and second processing unit can for example vary in their size, but can nevertheless be configured or combined with each other by the adapter plate. A processing system is preferably configured with various processing units that for example use various separation media or are responsible for various processing stages of a method. Equivalent processing units can be used when a capacity expansion is desired.

With respect to the fluid flow, the adapter plate is configured between the first processing unit and the second processing unit, and therefore fluidically connects the first processing unit and the second processing unit.

The adapter plate, which (at least partially) can be configured between two processing units in a modular processing system, permits making required adjustments regarding the fluid flow, which permit fluidically connecting processing units (in particular standard processing units) to connect to each other. This involves appropriately and at least partially rerouting or deflecting the fluid flow, or changing the direction of flow of the fluid flow, and/or the fluid flow can be regulated or controlled with the adapter plate, preferably or in particular its pressure, with which the fluid reaches the second processing unit. However, both adjustments can also be made simultaneously within one adapter plate.

At least one adapter plate can then be arranged between two sequentially arranged processing units in a processing system to make the required adjustments concerning the fluid flow.

Configuring process steps based on a modular processing system with at least one adapter plate between two processing units advantageously permits combining arbitrary process steps in a processing system such that the need for the footprint and system components can be reduced. The required investments are reduced accordingly. Additionally, in particular a compact and/or flexible configuration for the continuous operating mode in a processing system, including monitoring of several basic operations, is provided.

It is further preferred that at least one sensor is embedded in the first and second processing unit and/or in the adapter plate, in order to detect at least one property of the fluid flow in the first and second processing unit and the adapter plate.

This ensures continuous monitoring of the fluid flow over the course of the processing system. Using the measurement data of the sensors, the regulating or control device can analyze at least one parameter of the fluid flow at the various locations in the processing system, and can immediately initiate actions to correct the corresponding parameters of the fluid flow if a deviation from the predetermined limit values is identified. As a result, the processing result can be advantageously influenced during the course of the processing system, and parameters of the fluid flow that would result in a negative process result can be influenced in the desired manner. As already described above, without limitation, the pump and/or the at least one valve and/or the at least one deflection element in the adapter plate can be controlled or regulated accordingly by the control device.

The external control device is preferably coupled to the sensors in such a way that measurement data of the sensors can be read out, and the fluid flow in the processing units and or the adapter plate can be centrally controlled based on the read-out measurement data.

It is preferred that the at least one processing unit and/or the adapter plate each have at least one transponder that is designed to transmit measurement data of a corresponding sensor to the external control device; or wherein the sensors of the processing units and or the adapter plate are coupled to the external control device over a bus system.

A “transponder” is defined as a transmission unit that transmits the measurement data of the sensor wirelessly, optically, and/or by radio to the external control device. Alternatively, the processing system can comprise a bus system over which the at least one sensor can transmit measurement data to the external control device.

Preferably, the at least one deflection element and/or the pump and/or the at least one valve are controllable by an actuator.

An “actuator” is defined as a motion control unit that receives control commands from the external regulating or control device, and converts the commands into a mechanical motion. This can comprise actuating the at least one deflection element and/or the pump and/or the valve. The commands are output by the external control device.

Accordingly, not only are measurement data read out centrally, but the fluid flow can also be centrally controlled by automated means in the processing system.

The sensor can be designed to measure a pressure, a volumetric flow, a UV value, a pH value, a turbidity, and/or a viscosity of the fluid flow.

Accordingly, depending on need, at least one parameter of the fluid flow can be monitored, and any required adjustments can be made on the fluid flow. These can be commanded by the external control device and/or the external control device generates an output (such as an alarm) for the user. The latter can then make any further adjustments as needed, should for example the pH value of the fluid be too high.

Preferably, the at least one sensor, the at least one deflection element, the pump, and/or the at least one valve, each have a rechargeable battery.

A rechargeable battery provides the sensor with the required power to make measurements; the deflection element or the valve can if needed be readjusted with the power, and the power from the rechargeable battery allows the pump to generate the required pump output if required.

The rechargeable battery in particular has the advantage that cables do not need to be routed to the outside and do not need to be connected by the user. A rechargeable battery already supplies the respective component with sufficient power. The rechargeable battery can in particular also be recharged inductively.

Alternatively or additionally, the at least one sensor, the at least one deflection element, the at least one valve, and/or pump can have an umbilical power supply.

In particular when the processing system is operated for an extended time, an umbilical power supply has the advantage of supplying the required power for the aforementioned components.

The processing system preferably has a central power supply for the at least one sensor, the at least one deflection element, the at least one valve and/or the pump,

wherein the at least [ . . . ] processing unit and the at least one adapter plate have partial sections of the power supply that form the central power supply when assembled.

In other words, respectively one partial section of the umbilical power supply (cable) is integrated into the processing unit and in the adapter plate. When the processing system is assembled, the cable sections of the at least one processing unit and of the at least one adapter plate are connected to each other. As a result, power only needs to be supplied to the processing system at a single point. The central power supply, which extends through the processing system, allows all components in the processing system, such as the sensors, to be supplied with power.

The at least one sensor the at least one deflection element, the at least one valve, and the pump are preferably formed as single-use elements.

“Single-use” in this case means that the sensor, the deflection element, and the pump can be disposed after their use together with the processing unit or the adapter plate, depending on what element they are embedded into. As result, cleaning and reconditioning for further use in a new processing unit or adapter plate is avoided.

Additionally, the underlying task is solved by a method for centrally regulating a modular processing system for biopharmaceutical and/or chemical processes. The method comprises the steps:

Provide at least one processing unit;

Provide at least one adapter plate that can have at least one adapter channel through which at least one fluid flow can flow, wherein the adapter plate additionally has at least one deflection element and/or at least one valve and/or a pump;

Provide an external control device;

Directly or indirectly connect the adapter plate to the processing unit such that the fluid flow can flow from the adapter plate to the processing unit;

Detect at least one property of the fluid flow in the processing unit and/or the adapter plate using at least one sensor that is embedded in the processing unit and/or the adapter plate; and

Couple the external control device to the at least one sensor such that measurement data from the at least one sensor can be read out; and

Couple the external control device to the at least one deflection element and/or the pump and/or the at least one valve in the adapter plate such that the fluid flow in the processing unit and/or the adapter plate can be centrally controlled based on the read-out measurement data;

wherein the fluid flow can be at least partially deflected using the at least one deflection element in the adapter channel, and/or

wherein the fluid flow, preferably its pressure, can be regulated using the at least one valve and/or the pump in the adapter channel.

The processing system preferably comprises at least a first and a second processing unit; and

wherein the first and second processing unit are coupled to each other by the adapter plate such that the fluid flow can flow from the first processing unit to the second processing unit.

Respectively at least one sensor is preferably embedded in the first and second processing unit and in the adapter plate;

wherein the sensors detect at least one property of the fluid flow in the first and second processing unit and the adapter plate; and

wherein the sensors are coupled to the external control device such that measurement data of the sensors can be read out, and the fluid flow in the processing units and or the adapter plate can be centrally controlled based on the read-out measurement data.

These and other objectives, features, and advantages of the present invention become more evident by studying the following detailed description of preferred embodiments and the enclosed drawings. We also note that although embodiments are described separately, individual features of these embodiments can be combined to form additional embodiments.

### BRIEF DESCRIPTION OF THE DRAWINGS

FIG. 1a shows a basic construction of various processing units;

FIG. 1b shows a basic construction of various processing units;

FIG. 1c shows a basic construction of various processing units;

FIG. 1d shows a basic construction of various processing units;

FIG. 1e shows a basic construction of various processing units;

FIG. 1f shows a basic construction of various processing units;

FIG. 2a shows a processing system having two processing unit groups pursuant to an embodiment that are configured in parallel using an adapter plate;

FIG. 2b shows a processing system from FIG. 2a), having two processing unit groups that are configured in-series using an adapter plate;

FIG. 2c shows that the processing system from FIG. 2b, wherein the individual processing units are held together using termination brackets;

FIG. 3a shows a cross-section view through an adapter plate from FIGS. 2a) and 2b) with a multiway valve;

FIG. 3b shows the multiway valve from FIG. 3a);

FIG. 4a shows a cross-section view of a two-part adapter plate having a sensor pursuant to a further embodiment;

FIG. 4b shows a perspective view of the adapter plate from FIG. 4a);

FIG. 5a shows a cross-section view of an adapter plate pursuant to a further embodiment having an auxiliary branch into which an adapter plate sensor is integrated;

FIG. 5b shows a perspective view of the adapter plate from FIG. 5a);

FIG. 6a shows a processing system having an adapter plate pursuant to a further embodiment that has two auxiliary outlets or auxiliary inlets respectively;

FIG. 6b shows an embodiment of an adapter plate from FIG. 6a), wherein an auxiliary inlet is used for fluid dilution;

FIG. 6c shows an embodiment of an adapter plate from FIG. 6a), wherein auxiliary inlets are used for virus inactivation;

FIG. 7 shows a processing system having an adapter plate pursuant to a further embodiment into which a pump is integrated;

FIG. 8a shows a cross-sectional view through an adapter plate having a piston pump in an intake position;

FIG. 8b shows a cross-section view of the adapter plate from FIG. 8a) in a stroke position;

FIG. 9a shows an exploded view of an adapter plate having a peristaltic pump pursuant to a further embodiment;

FIG. 9b shows a cross-section view of the adapter plate from FIG. 9a);

FIG. 10a shows a processing unit pursuant to an embodiment into which a sensor is embedded whose data are transmitted wirelessly to an external control unit; and

FIG. 10b shows a processing system pursuant to an embodiment with sensors that are transmitted to the external control device over a bus system.

### DETAILED DESCRIPTION

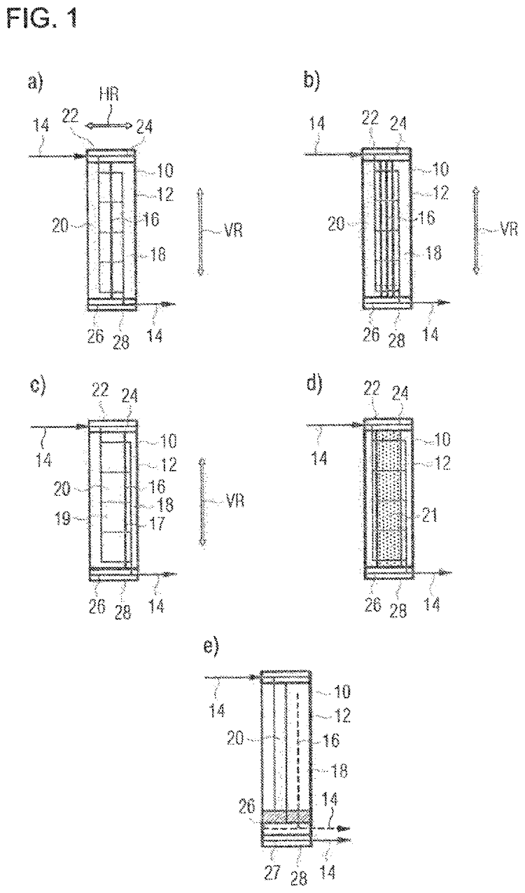

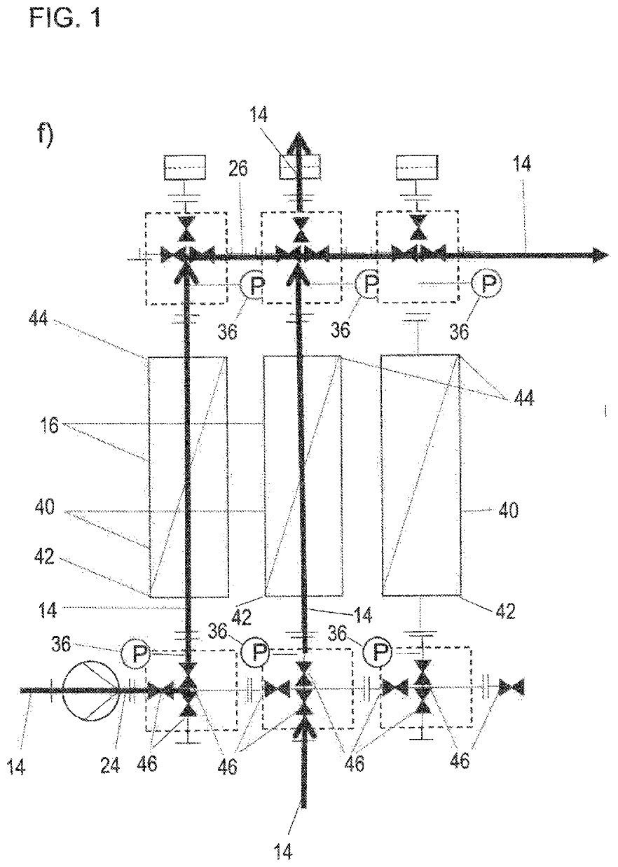

Various processing units are used for biopharmaceutical and chemical processes, wherein said processing units can be used in the context of the present invention. FIGS. 1a) to 1e) show a basic construction of various processing units that can be used in the context of the present invention. These represent a selection and are not an exhaustive list.

FIG. 1a) shows a processing unit 10 that can be used to perform a certain filtration step in a biopharmaceutical or chemical process. For this purpose, the processing unit 10 has a processing housing 12 through which a fluid flow 14 can flow. The fluid flow 14 comprises the medium to be filtered. At least one filter medium 16 is arranged in the processing housing 12, the filter medium 16 comprising a porous material that is selected or used based on what particles or what substances are to be filtered out from the fluid flow 14 by the processing unit 10. The filter medium 16 can for example be a virus filter, a sterile filter, a depth filter, or a membrane absorber. The filter medium 16 is preferably formed as a filter mat or filter membrane or filter membrane layer(s). In a preferred embodiment, the filter medium 16 can consist of several layers. Typically, the filter medium 16 is arranged substantially in vertical direction VR in the processing housing 12. In the processing housing 12, the filter medium 16 separates a filtrate side 18 from a retentate side 20. The filter medium 16 is fluidically permeable, wherein filter medium-specific substances cannot pass through the filter medium 16. Because the fluid flow 14 is as specified directed from the retentate side 20 to the filtrate side 18, these filter medium-specific substances remain on the retentate side 20 and/or in the filter medium 16, but substantially do not reach the filtrate side 18 of the processing unit 10. A fluid pressure differential exists between the retentate side 20 and the filtrate side 18 as a function of the applied fluid pressure and/or the permeability of the filter medium 16.

At least one inlet channel 24 is located at a preferably upper end 22 of the processing housing 12. The inlet channel 24 preferably extends in substantially horizontal direction HR and feeds the processing unit 10 with the medium to be filtered. As indicated in FIG. 1 by an arrow, the fluid flow 14 flows into the processing housing 12 over the inlet channel 24. In FIG. 1 this means that a fluid flow 14 flows from the left into the processing housing 12. At least a part of the fluid flow 14 subsequently flows from the retentate side 20 through the filter medium 16 to the filtrate side 18. If the processing unit 10 is configured in parallel with a further processing unit (not shown here), a further part of the fluid flow 14 flows directly to the further processing unit without permeating the filter medium 16. This means, that this part of the fluid flow 14 flows into the inlet channel 24 of the further (not shown) processing unit. The fluid flow 14 that permeated the filter medium 16 (“filtrate”) subsequently flows into an outlet channel 26 at the preferably bottom end 28 of the processing housing 12, and from there flows out of the processing housing 12.

The outlet channel 26 likewise preferably extends in substantially horizontal direction HR in the processing housing 12. The filtrate that exits the processing housing 12 can subsequently flow into the outlet channel 26 of a further processing unit (not shown here) (parallel arrangement) and/or for further processing into the inlet channel 24 of the further processing unit (in-series arrangement).

FIG. 1b) shows the processing unit 10 from FIG. 1a), which merely differs from the latter in that the filter medium 16 has a multilayered design.

FIG. 1c) shows a processing unit 10 that is principally formed similar to the processing unit 10 from FIG. 1a), but differs in the type of filtration. The following therefore only describes the parts of the processing unit 10 from FIG. 1c) that differ from the processing unit 10 from FIG. 1a).

Specifically, the processing unit from FIG. 1c) for formed for precoat filtration. The filter medium 16 is for this purpose formed as a precoat filter. The filter medium 16 in this case comprises a filter carrier 17 that is preferably arranged in vertical direction VR in the processing housing 12, and that has a relatively coarse design. Prior to permeating the filter, the pre-coat medium is typically mixed with the fluid. This enables the building up a filter cake (not shown here). The filter carrier 17 is selected such that at least one filter auxiliary agent is retained. A vacant space 19 is formed on the retentate side 20 to provide a corresponding space for the filter cake in the processing housing 12.

FIG. 1d) shows a further processing unit 10. The latter has a design similar to the processing unit 10 from FIG. 1a) but comprises a bulk medium 21 instead of a filter medium 16. The bulk medium 21 can specifically be gels or activated carbon, such that the processing unit 10 from FIG. 1c) is suited for chromatography.

Substance mixtures can be separated in the context of chromatography.

The bulk medium 21 in this case acts as a stationary phase that is immovably arranged in the processing unit 10. Using a mobile phase (such as water), a substance mixture is transported to the stationary phase. Due to an interaction of the stationary phase with the individual substances in the mobile phase, the flow-through time of the corresponding substance through the processing unit 10 can be delayed such that substances can be separated.

FIG. 1e shows a further processing unit 10. The latter likewise has a design similar to the processing unit 10 from FIG. 1a), but differs in that a second outlet channel 27 is provided. As a result, this processing unit 10 is suited for crossflow filtration. This involves pumping a suspension to be filtered at a high speed in parallel to the filter medium 16 and extracting the filtrate transverse to the direction of flow. The filtrate can then be extracted over one of the outlet channels 26. The part of the fluid flow 14 that does not permeate the filter medium 16 (or the retentate), can be extracted from the processing unit 10 over the second outlet channel 27. Depending on need, the filtrate or the retentate from the crossflow filtration can then be processed further in the context of processing systems described below.

Whereas the filter medium 16 in FIGS. 1a) to 1e) is formed as a flat filter, the filter medium 16 can alternatively be a filter cartridge or filter capsule 40, as shown in FIG. 1f).

Whereas flat filters have a flat design and form a plane, a filter cartridge 40 has a cylindrical shape. The filter medium 16 preferably comprises a pleated filter material arranged in multiple layers to form the cylindrical filter cartridge 40. In order to ensure the required stability of the filter cartridge 40, the filter cartridge 40 preferably comprises a cylindrical support core that braces the filter medium 16 from the inside, and a rigid outer cage that envelops the filter medium 16 from the outside and braces the latter from the outside. The support core and the outer cage are however both designed such that a fluid flow is permitted through these.

FIG. 1f) generally shows a schematic representation of a fluid flow 14 through the filter cartridge 40. The fluid flow 14 can in this case be fed such that the fluid flow 14 flows from an inner side of the filter cartridge 40 to an outer side of the filter cartridge 40, or from the outer side of the filter cartridge 40 to the inner side of the filter cartridge 40. The feed direction is in this case influenced by an end cap that seals the cylindrical body of the filter cartridge 40 at an end side.

If the flow is to be directed through the filter cartridge 40 from the interior to the exterior, the fluid flow 14 is fed from a first side 42 of the filter cartridge 40 to an interior of the filter cartridge 40. But in this case, the second, opposing side 44 of the filter cartridge 40 is sealed by the end cap such that the fed fluid is forced to flow through the filter medium 16 of the filter cartridge 40 from the interior to the exterior.

If the flow is alternatively to be directed through the filter cartridge 40 from the exterior to the interior, the filter cartridge 40 is sealed on the first end 42 by an end cap such that fluid flows from the exterior to the interior. By contrast, the second and 44 of the filter cartridge 40 is open this case.

The filter cartridge 40 itself is inserted or installed in a flat processing housing 12. The processing housing 12 preferably has walls in its interior that appropriately route the fluid flow 14 such that the fluid flow 14 of the filter cartridge 40 is fed in the desired manner. If the fluid flow 14 for example is to be routed from the interior to the exterior on the filter cartridge 40, walls can be arranged that route a fluid flow 14 to the interior of the filter cartridge 40. As a result, any contact of the medium to be filtered with an exterior of the filter cartridge 40 can be avoided. This also avoids that the filtered medium comes into contact with the medium to be filtered.

If the fluid flow 14 is to flow from the exterior to the interior on the filter cartridge 40, a corresponding wall is preferably likewise arranged adjacent to the second side 44 of the filter cartridge 40. The latter prevents that the filtered medium that exits on the second side 44 of the filter cartridge 40 comes into contact with the medium to be filtered on the exterior of the filter cartridge 40.

The filter cartridge 40 can be installed in the processing housing 12 in a vertical or horizontal orientation. The inlet channel 24 over which the medium to be filtered flows into the processing housing 12 is in this case appropriately formed such that a flow can appropriately flow through the filter cartridge in relation to its vertical or horizontal orientation. The same applies for the outlet channel 26 over which the filtered fluid can flow out of the processing housing 12.

As shown in FIG. 1f), several filter cartridges 40 can be arranged in parallel in a processing housing 12. Feeding and extracting the fluid flow of 14 from and to the respective filter cartridge 40 is accomplished as was already described above in relation to a filter cartridge 40. However, an additional partition wall can be arranged between the individual filter cartridges 40 such that the filter cartridges 40 are separated from each other.

At least one filter cartridge valve 46 can be arranged in the inlet channel 24 and/or in the outlet channel 26. In the inlet channel 24, the filter cartridge valve 46 can be designed to facilitate or to prevent the flow to continue into the inlet channel 24. A filter cartridge valve 46 can further be designed to facilitate or to block the fluid flow 14 from flowing toward or into a filter cartridge 40. As shown in FIG. 1f), a filter cartridge valve 46 can also be designed to grant access from the exterior into the inlet channel 24 such that additives or a further fluid flow 14 can flow into the inlet channel 24.

In the inlet channel 26, the filter cartridge valve 46 can be designed to facilitate or to prevent the flow to continue into the outlet channel 26. As shown in FIG. 1f), a filter cartridge valve 46 can also be designed to grant an additional outlet from the outlet channel 26 such that at least a part of the fluid flow 14 can flow from the outlet channel 26.

Filter cartridge valves 46 are advantageous in particular for integrity tests. Using the filter cartridge valves 46, filter cartridges 40 can separately undergo an integrity test.

At least one processing unit sensor 36 can be embedded into the processing housing 12. The latter is positioned such that a desired parameter of the fluid flow 14 can be monitored or measured in the processing unit 10. The associated processing unit sensor 36 is described in further detail below.

For the integrity test, the processing unit sensor 36 can in particular be designed to detect the pressure of the fluid flow 14, as shown in FIG. 1f). Advantageously, a processing unit sensor 36 is located upstream and downstream of a filter cartridge 40 such that a pressure differential of the fluid flow 14 can be detected.

Filter cartridges 40 are particularly suitable in particular for filtration applications wherein high pressures act on the filter medium 16.

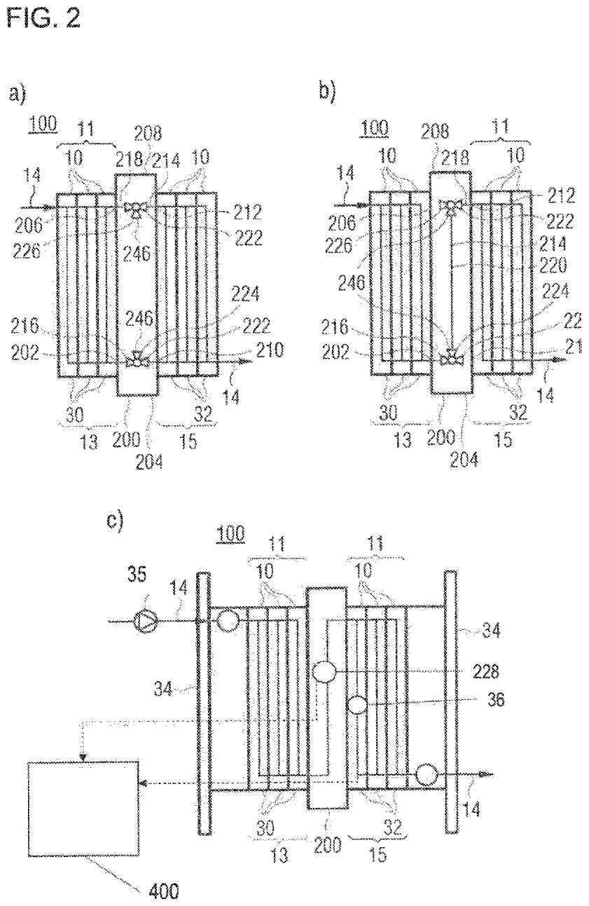

FIGS. 2a) and 2b) each show processing systems 100 having a plurality of the processing units 10 described in FIGS. 1a) to 1e). Processing units 10 that are coupled directly to each other form a processing unit group 11. As shown in FIGS. 2a) and 2b), the processing systems 100 each comprise a first processing unit group 13 and a second processing unit group 15 that are coupled by an adapter plate 200 pursuant to an embodiment of the invention. The processing units 10 coupled directly in FIGS. 2a) and 2b) are shown as units configured in parallel, but can also be configured in series. The adapter plate 200 allows the two processing unit groups 11, which are coupled by the adapter plate 200, to be configured in parallel (see FIG. 2a)) and also in-series (see FIG. 2b)). Optionally, the adapter plate 200 has at least one deflection element (described below) by which these configurations are switched. This is readily accomplished in the processing system 100 without design changes. By simply switching one or several deflection elements, the processing unit group 11 configuration can be switched from a parallel to an in-series configuration.

FIG. 2a) shows a parallel configuration of processing unit groups 11 that are coupled by the adapter plate 200. FIG. 2b) shows an in-series configuration of processing unit groups 11 that are coupled by the adapter plate 200. Although the FIGS. 2a) and 2b) show the processing unit groups 11 coupled by the adapter plate 200, the adapter plate 200 can also be used to couple only one processing unit 10 to a processing unit group 11 or to individual processing units 10 to each other.

The adapter plate 200 is preferably substantially formed as a plate, and can accommodate a fluid flow 14. The adapter plate 200 is preferably formed as a single-use component, wherein the material properties are preferably selected such that a sterilization method, such as gamma irradiation, autoclaving, purging with gas such as ethylene oxide and/or hot steam, can be used. The adapter plate can in particular be produced from plastics. Processing units 10 that are arranged in flow direction upstream of an adapter plate 200 are referred to as first processing units 30, whereas processing units 10 data are arranged in flow direction downstream of an adapter plate 200 are referred to as second processing units 32. In other words, the first processing unit group 13 consists of first processing units 30, and the second processing unit 15 consists of two processing units 32, as shown in FIGS. 2a) and 2b). The adapter plate 200 is arranged between the first processing unit group 13 and the second processing unit group 15, and connects these fluidically. The adapter plate 200 comprises at least one inlet opening through which the fluid flow 14 can flow into the adapter plate 200. As shown in FIG. 2a), the adapter plate 200 has two inlet openings, that is to say a first inlet opening 202 preferably at the bottom end 204 of the adapter plate 200, which is coupled to the outlet channel 26 of the first processing unit 30 that is arranged directly upstream of the adapter plate 200, and a second inlet opening 206 preferably at the upper end 208 of the adapter plate 200 that is coupled to the inlet channel 24 of this first processing unit 30. In other words, a fluid flow 14 can flow from the aforementioned first processing unit 30 over the first and second inlet opening 202 and 206 into the adapter plate 200. The adapter plate 200 also has at least one outlet opening through which the fluid flow 14 can exit from the adapter plate 200. As shown in FIG. 2a), the adapter plate 200 has two outlet openings, that is to say a first outlet opening 210 preferably arranged at or near a bottom end 204 of the adapter plate 200 and which is or can be coupled to the outlet channel 26 of the second processing unit 32 that is arranged directly downstream of the adapter plate 200, and a second outlet opening 212 preferably arranged on or near an upper end 208 of the adapter plate 200 and which is or can be coupled to the inlet channel 24 of this second processing unit 32. An at least one adapter channel 214 extends within the adapter plate 200 and fluidically connects the inlet and outlet openings to each other. The adapter channel 214 specifically has a first channel area 216 that extends between the first inlet opening 202 and the first outlet opening 210. A second channel area 218 extends between the second inlet opening 206 and the second outlet opening 212. The first and second channel area 216 and 218 is fluidically connected by a connecting channel area 220.

The construction of the adapter channel 214 described above is in particular advantageous when the at least one deflection element can be used to switch between an in-series and parallel configuration of two processing units 10. If the adapter plate 200 does not have a deflection element, only a predetermined fluid path can then be specified in the adapter plate 200. Alternatively, the adapter channel 214 can be constructed as described above, but sections of the adapter channel 214 are permanently sealed, resulting in a predetermined fluid path through the adapter plate 200.

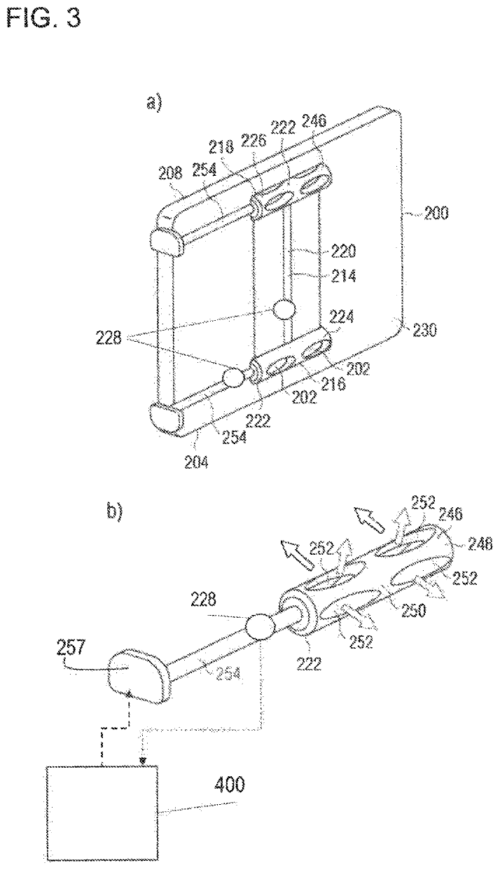

As shown in FIGS. 2a) and 2b), at least two deflection elements are arranged in the adapter channel 214. Specifically, these are deflection valves 222. In a preferred embodiment, a first deflection valve 224 is arranged in the first channel area 216, and a second deflection valve 226 is arranged in the second channel area 218. In a concrete embodiment, the deflection valves 222 can be formed as multiway valves 246. FIGS. 2a) and 2b) show a related example of a three-way valve and a first valve 224 and a second valve 226. An alternative embodiment of a multiway valve is described as follows based on FIGS. 3a) and 3b).

The at least one first and second processing unit 30 and 32 can have an identical design or can be different in order to perform different process types. The first processing units 30 in the first processing unit group 13 and the second processing units 32 of the second processing unit group 15 preferably have the identical design. However, it is also conceivable that the first processing units 30 and the second processing units 32 respectively differ from each other. For example, the at least one first processing unit 30 can be formed as a depth filter, whereas the at least one second processing unit 32 can be formed as a sterile filter.

FIG. 2c) shows the processing system 100 from FIG. 2b), wherein the individual processing units 10 and the adapter plate 200 are held together by termination brackets 34 (multiway valves 246 are not shown here). In other words, respectively one termination bracket 34 is located at the entry point at which the fluid flow 14 enters the processing system 100, and at the outlet point at which the fluid flow 14 exits the processing system 100. The latter can preferably be formed as a termination plate, such that the individual processing units 10 are held between these termination plates like a sandwich. Inlets or outlets can be part of the termination bracket 34. A further termination plate with corresponding connections can in particular also be incorporated between a termination bracket 34 and the respectively adjacent processing unit 10. The fluid flow 14 is preferably pumped into the processing system 100 by an inlet pump 35. FIG. 2c) shows an inlet pump 35 that is arranged upstream of the termination bracket 34 in regards to the direction of flow. The inlet pump 35 can advantageously also be in the adapter plate 200 located between the termination bracket and the adjacent processing unit 10. A space-saving design can be achieved in this way.

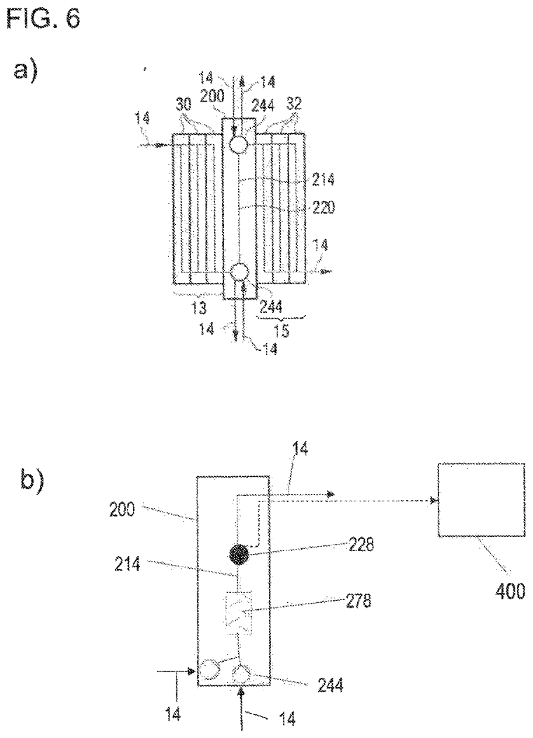

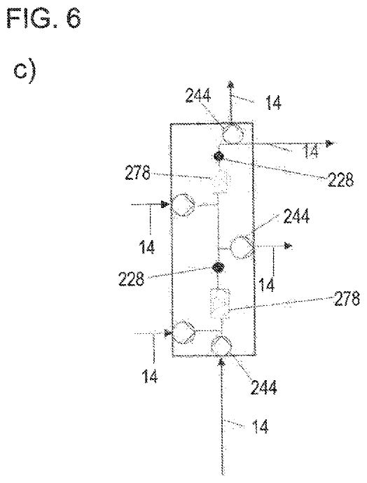

Although all processing systems 100 shown in FIGS. 2a) to 2c) comprise adapter plates 200 that are arranged between two processing units 10 in order to fluidically connect these, this arrangement is not mandatory. The adapter plate 200 can merely be configured upstream of at least one processing unit 10 in order to feed a fluid to the at least one processing unit 10. The adapter plate 200 can in particular be arranged between the termination bracket 34 and a processing unit 100. This is for example advantageous when the processing unit 10 configured downstream is a unit for membrane chromatography. In the context of chromatography, various media are typically applied sequentially onto the filter medium 16. (The steps sanitizing, conditioning, rinsing, equilibrating can for example be performed prior to loading with the protein solution actually to be cleaned. The various inlets on the adapter plate 200 can therefore be used to feed of the individual media to the processing unit 10. After loading with the medium to be cleaned, a washing step and an elution step can then follow. Here as well, separate inlets and/or outlets on the adapter plate 200 are helpful, as is for example described in relation to FIG. 6a). Respectively one deflection element, by which the configuration can be “toggled” between inlets and/outlets, can be arranged for this purpose on such an inlet and/or outlet).

The processing system 100 according to the invention further has at least one sensor that is embedded in at least one processing unit 10 and/or at least one adapter plate 200. For the embodiment of FIGS. 2a) to 2c), FIG. 2c) shows an example of an adapter plate sensor 228 that is embedded in the adapter plates 200, and a processing unit sensor 36 that is embedded in the processing unit 10 arranged downstream of the adapter plate 200 in the direction of flow.

The at least one adapter plate sensor 228 is in particular embedded into the adapter plate 200 such that the adapter plate sensor 228 at least partially reaches into the adapter channel 214 in order to make contact with the fluid flow 14 and to take the desired measurements on the fluid.

The adapter plate sensor 228 can be formed such that for example the pressure, the volumetric flow, the UV value, the pH value, the turbidity, and/or the viscosity of the fluid flow 14 can be measured. The measured values can in turn be used to control or regulate the filtration process. The measured values are for this purpose transmitted to the external control device 400. The adapter plate sensor 228 can be formed as a cost-effective single-use component such that the adapter plate sensor 228 can be disposed together with the adapter plate 200 after their use. The adapter plate sensor 228 can in particular already be integrated by the factory into the adapter plate 200. The adapter plate 200 is then already shipped with a calibrated and sterilized adapter plate sensor 228.

The shown adapter plate 200 couples two processing units 10, in-series here. However, it is also possible to use adapter plate sensors 228 for a parallel configuration of two processing units 10. However, it is advantageous in this case that the adapter plate sensor(s) 228 is/are arranged in or on the first and/or second channel area 216 and 218 in order to establish contact to the fluid flow 14. If two processing units 10 are configured in-series, the adapter plate sensor 228 can also be arranged in or on the connecting channel area 220.

At least one adapter plate sensor 228 can also be specified on at least one deflection element in the at least one adapter plate 200. This adapter plate sensor 228 is designed to detect the physical setting of the deflection element and to transmit this state to the external control device 400.

The at least one processing unit sensor 36 can detect at least one parameter of the fluid flow 14 in the corresponding processing unit 10, as was already described with regard to the adapter plate sensor 228. The processing unit sensor 36 is in particular embedded into the processing unit 10 such that the latter makes at least partial contact with the fluid flow 14 in order to take corresponding measurements. The measured values can likewise be transmitted to the external control device 400.

The processing unit sensor 36 can be arranged at any arbitrary position in the processing unit 10 to take the desired measurement. The processing unit sensor 36 can for example be arranged on the filtrate side 18 of the processing unit 10 in order to advantageously determine or monitor the filtration result of a processing unit 10. The processing unit sensor 36 is in particular preferably also formed as a cost-effective single-use component such that the processing unit sensor 36 can be disposed together with the processing unit 10 after its use.

As shown in FIG. 2c), at least one processing unit sensor 36 is preferably located in the processing unit 10 that is configured downstream of the adapter plate 200. This processing unit sensor 36 can for example monitor what pressure the fluid flow 14 exerts on the filter medium 16. But processing unit sensors 36 can also be specified in any other of the remaining processing units 10 of the processing system 100.

FIG. 3a) shows a cross-section view of an adapter plate 200. As shown in FIG. 3a), the adapter plate 200 is preferably formed as two parts and comprises an inlet plate 230 and an outlet plate (not shown here). The plate of the adapter plate 200 in which the at least one inlet opening is arranged is referred to as inlet plate 230. The plate of the adapter plate 200 in which the at least one outlet opening is arranged is referred to as outlet plate. The shape and/or size of the inlet plate 230 and the outlet plate can be identical. Respectively one multiway valve 246, which acts as a deflection element, is located in the first and/or second channel area 216 and 218. The multiway valve 246 comprises a valve tube 248 that respectively extends at least partially in the first and second channel area 216 and 218, where it is positioned so as to be actuatable (in particular such that it can be rotated or turned). An actuation (in particular rotation) can be accomplished by a deflection actuator 257. The deflection actuator 257 can in this case receive actuation commands from the external control device 400 and can convert these into a mechanical actuation of the multiway valve 246. The deflection actuator 257 can in particular be arranged on an actuation arm 254 that protrudes from the adapter plate 200 (see FIGS. 3a) and 3b)). As shown in FIGS. 3a) and 3b), at least one adapter plate sensor 228 can be arranged on at least one of the multiway valves 246 to detect the physical setting of the multi-way valve 246 and to transmit this information to the external control device 400. The adapter plate sensor 228 can in particular be arranged on the actuation arm 254 of the multiway valve 246.

At least two valve openings 252 are formed in a jacket surface 250 of the valve tube 248. In relation to the actuation direction (in particular the direction of rotation), these are arranged at an offset to each other such that a first fluid flow or a second fluid flow is enabled in a first setting of the valve tube 248. This means that for example the first fluid flow enters into the valve tube 248 over a first inlet opening 202 of the adapter plate 200 through a first valve opening 252 that at least partially overlaps with the first inlet opening 202.

The second valve opening 252 at least partially overlaps the first outlet opening 210 such that the first fluid flow can flow from the adapter plate 200 over the first outlet opening 210. This applies accordingly for the second fluid flow and the second inlet and outlet opening 206 and 212. A fluid flow 14 through the connecting channel area 220 is blocked in the respective channel areas based on the physical setting of the valve openings 252. But because the first and second fluid flow through the multiway valves 246 is permitted, the first and second processing unit 30 and 32, which are each directly coupled to the adapter plate 200, are configured in parallel to each other.

The valve tubes 248 however preferably each have at least three valve openings 252, as shown in FIGS. 3a) and 3b). These are arranged such that in a first alignment (in particular a rotational physical setting) of the valve tube 248, at least two valve openings 252 are aligned such that the first and second fluid flows described above are permitted, and that therefore the first and second processing unit 30 and 32, which are each directly coupled to the adapter plate, are configured in parallel. However, a fluid flow 14 through the connecting channel area 220 is blocked because no valve opening 252 at least partially overlaps with the connecting channel area 220.

Based on the arrangement of the at least three valve openings 252, either the pathway to an inlet opening or to an outlet opening are blocked in a second alignment (in particular a rotational physical setting) of the respective valve tube 248, whereas access to the connecting channel area 220 is permitted. This permits an in-series configuration of the first and second processing unit 30 and 32.

In other words, the multiway valves 246 must be aligned in an in-series configuration such that a fluid flow 14 can enter into the first valve tube 248 over the first inlet opening 202 of the adapter plate 200 and over a valve opening 252 that at least partially overlaps with the first inlet opening 202. The fluid flow 14 can flow into the connecting channel area 220 over a valve opening 252 that at least partially overlaps with the connecting channel area 220. The pathway to the first outlet opening 210 is blocked by the valve tube 248 because no valve opening 252 overlaps with the first outlet opening 210. The fluid flow 14 can then flow into the second valve tube 248 over a valve opening 252 of the second valve tube 248 that at least partially overlaps with the connecting channel area 220. The fluid flow 14 can flow out of the adapter plate 200 over a valve opening 252 in the second valve tube 248 that at least partially overlaps with the second outlet opening 212 of the adapter plate 200. The pathway to the second inlet opening 206 is blocked by the valve tube 248 because no valve opening 252 overlaps with the second inlet opening 206.

A switch between an in-series and parallel configuration of two processing units 10 or processing unit groups 11 can then be easily made without design-based reconfiguration actions. The adapter plate 200 is in particular designed as a compact assembly component such that the processing system 100 has a small footprint. The deflection valves 222 can be designed as cost-effective single-use components. Potential applications include: Switching over tangential flow filtration from standard mode to single pass, switching between parallel and in-series chromatography unit configurations, for example for in-series combinations of different chromatography media or to improve capacity utilization.

FIGS. 3a) and b) describe an exemplary embodiment of a rotating deflection element. The physical setting of the deflection element can be actuated by control signals from the external control device 400. We note that this is however possible for any other type of deflection element (see for example the three-way valve described above in FIGS. 2a) to 2c)).

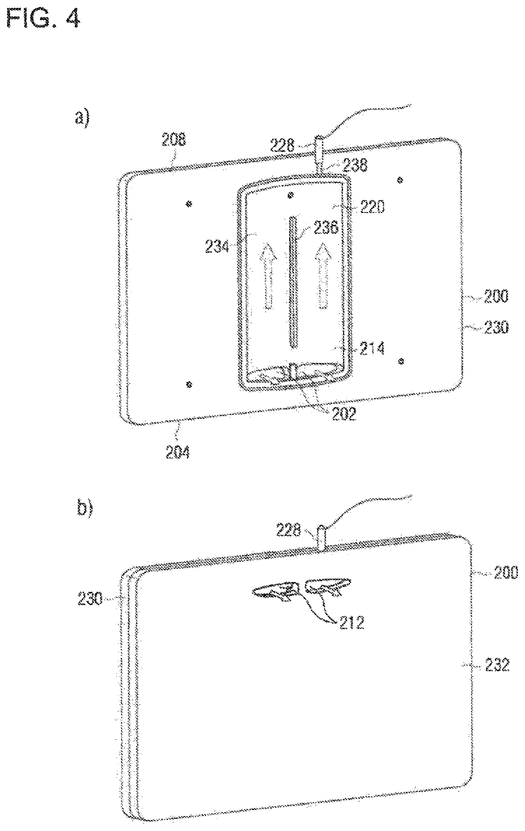

FIGS. 4a) and 4b) show a preferred embodiment of an integration of at least one adapter plate sensor 228 and an adapter plate 200. As shown in FIG. 4b), the adapter plate 200 is likewise preferably formed in two or multiple parts. The inlet plate 230 comprises the at least one first inlet opening 202, preferably at the lower end 204 of the adapter plate 200. As shown in FIG. 4a), the inlet plate 230 comprises two first inlet openings 202. However, it does not have any second inlet opening(s) 206. Because the shown adapter plate 200 is exclusively intended to allow an in-series configuration of two processing units 10, second inlet openings 206 is also not mandatory. For a parallel configuration, at least a second inlet opening 206 could be formed in the inlet plate 230, preferably at the upper end 208 of the adapter plate 200.

As also shown in FIG. 4b), the outlet plate 232 comprises at least one second outlet opening 212. In the specific case in FIG. 4b), the outlet plate 232 has two outlet openings 212. If a parallel configuration of two processing units 10 is desired, the outlet plate 232 can additionally have at least one first outlet opening 210. The inlet and outlet plate 230 and 232 can be threaded and or glued and or welded and or clicked together.

A corresponding channel depression 234 can be formed in the inlet and or outlet plate 230 and 232. If the inlet and outlet plate 230 and 232 are assembled together, an adapter channel 214 is formed that allows a fluid flow 14 to flow through the adapter plate 200. At least one flow web 236 is preferably formed in the connecting channel area 220 of the adapter channel 214, said flow web 236 preferably extends substantially in flow direction of the fluid flow 14 and contributes toward guiding the fluid flow 14 in an improved manner. The adapter plate sensor 228 can be integrated into the inlet and/or outlet plate 230 and 232 and can protrude into the adapter channel 214 such that a measurement element of the adapter plate sensor 228 can make contact with the fluid flow 14. The adapter channel 214 preferably at least sectionally comprises a connecting channel 238 to the adapter plate sensor 228. This connecting channel 238 can be an at least sectional depression in the adapter channel 214 into which the sensor 228 protrudes at least partially.

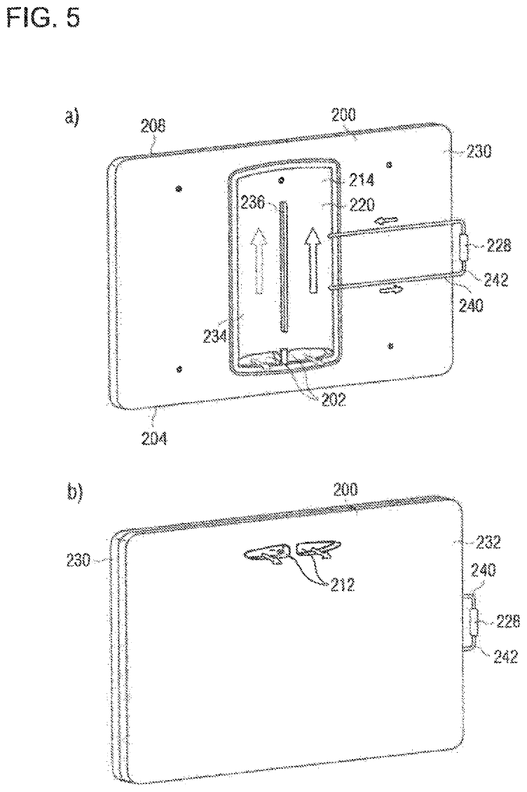

FIGS. 5a) and 5b) show the adapter plate 200 from the FIGS. 4a) and 4b), but in this embodiment, the adapter plate sensor 228 is in or on an auxiliary branch 240. The auxiliary branch 240 represents a branch from the adapter channel 214 into which at least a part of the fluid flow 14 is redirected. This part of the fluid flow 14 flows for at least a certain time from the adapter channel 214 in order to be preferably routed back into the adapter channel 214 at a later point.

The auxiliary branch 240 can be used to branch off small quantities of the fluid flow 14 for corresponding measurements. The auxiliary branch 240 can be formed with a separate channel element 242 that is at least partially integrated into the adapter plate 200. As shown in FIGS. 5a) and 5b), a part of the auxiliary branch 240 can protrude from the adapter plate 200. As a result, in particular the adapter plate sensor 228 can also be arranged outside of the adapter plate 200. However, in this arrangement, the adapter plate sensor 228 is nevertheless also regarded as being “embedded” into the adapter plate 200.

FIG. 6a) shows a processing system 100 having a first and second processing unit group 13 and 15 that are coupled by an adapter plate 200. In the shown embodiment, the linked processing unit groups 11 are configured in-series by the adapter plate 200; it is however likewise possible to configure the two processing unit groups 11 in parallel pursuant to the other described embodiments. As described above, the adapter plate 200 can also be formed as only an upstream adapter plate 200.

Pursuant to FIG. 6a), at least one auxiliary outlet and/or inlet 244 can be formed in the adapter plate 200. At least a part of the fluid flow 14 can be extracted from, or flow out of, the adapter channel 214 over an auxiliary outlet. The extracted fluid flow 14 can flow back into the adapter channel 214 or access to the adapter channel 214 can be provided by an auxiliary inlet. Use of at least one auxiliary outlet or inlet for example facilitates the external addition of a pump and integration tests on different processing units 10 of the processing system 100, the evacuation of the fluid from the first or further upstream processing units 30 for temporary buffering or for further processing, venting and/or sampling. In particular, at least one diafiltration medium and or other reagents can be added over an auxiliary inlet. FIG. 6a) shows an adapter plate 200 having two auxiliary outlets and inlets 244. Tri-clamps or sterile connectors can be potential connections on an auxiliary outlet and auxiliary inlet 244. In order to feed or evacuate the fluid on the auxiliary outlet or inlet 244 respectively, an auxiliary pump or an auxiliary valve can be appropriately specified on the auxiliary outlet or inlet 244.

FIG. 6b) shows an embodiment of an adapter plate 200 having at least one auxiliary inlet 244 that can be used to add a further fluid for diluting the fluid that is already present in the adapter plate 200. The further fluid can be mixed with the fluid already present in the adapter plate 200 by a static mixer 278. An exemplary application that requires a dilution is the requirement to reduce the salt concentration in the fluid during a partial step of the antibody-polishing method.

A “static mixer” 278 is defined as a device for mixing fluids wherein the mixing action is strictly achieved by fluid motion. In order to achieve a mixing fluid motion, flow-influencing elements are provided in the adapter channel 214. Such elements can be sequentially arranged screw, fin, or grid-shaped elements. The fluids to be mixed are jointly added to the mixer 278 in the desired mixing ratio. The elements split the substance flow, twist the flows and reunite them such that the desired mixing is achieved.

In the specific case of the present adapter plate 200, a fluid flow 14 is routed into the adapter plate 200. The fluid flow 14 can for example originate from an upstream processing unit 10. In order to now dilute this fluid flow 14 in the adapter plate 200, a further fluid flow 14 can be routed into the adapter plate 200 over an auxiliary inlet 244. These two fluid flows 14 are then mixed by the static mixer 278.

The adapter plate 200 preferably comprises at least one adapter plate sensor 228 that is designed to monitor the dilution step. For this purpose, the adapter plate sensor 228 is preferably arranged in flow direction downstream of the static mixer 278. The values detected by the adapter plate sensor 228 can subsequently be transmitted to the external control device 400. Based on the received measurement data, the latter can for example regulate an auxiliary pump on the auxiliary inlet 244 to regulate the quantity of the dilution fluid to be added.

If the degree of dilution does not have the required value, the fluid flow 14 could preferably also be redirected over an auxiliary outlet 244. The redirected fluid flow could then be directed back to be diluted further or to be discarded entirely. Only a fluid flow 14 having the desired dilution can subsequently be directed to the connected processing unit 10.

FIG. 6c) shows a further embodiment of an adapter plate 200 having at least one auxiliary outlet or inlet 244. In this case, the adapter plate 200 can in particular be used for virus inactivation.

The arrangement is principally similar to the one in FIG. 6b). However, instead of a dilution solution, a fluid for reducing the pH is instead added over an auxiliary inlet 244. Using the static mixer 278, the pH reducer is mixed with the fluid flow 14 flowing through the adapter plate 200.

Similar to FIG. 6b), at least one adapter plate sensor 228 can be located downstream of the static mixer 278. The adapter plate sensor 228 in this case preferably detects the pH value of the fluid flow 14 downstream of the static mixer 278. The measured values are then transmitted to the external control device 400. If the pH value is too high, the fluid flow 14 can be redirected out of the adapter plate 200 over an auxiliary outlet 244. The redirected fluid flow 14 is either directed back into the adapter plate 200, and the pH value is reduced further, or the fluid is discarded. Based on the received measurement data, the external control device 400 can for example regulate an auxiliary pump on the auxiliary inlet 244 to regulate the quantity of the pH reducer to be added.

If the fluid has a suitable pH value, the fluid can continue to flow in the adapter plate 200. In order to again increase the pH value, a further static mixer 278 is subsequently located therein. A fluid for increasing the pH value is added over a further auxiliary inlet 244, and mixed in the adapter plate 200 with the fluid flow 14 using a further static mixer 278. At least one further adapter plate sensor 228 located downstream of the further static mixer 278 preferably in turn monitors the achieved pH value of the fluid and transmits the measurement data to the external control device 400. As was already the case with the preceding static mixer 278, the defective fluid can here as well either be redirected or discarded over a further auxiliary outlet 244. Based on the received measurement data, the external control device 400 can for example regulate an auxiliary pump on the auxiliary inlet 244 to regulate the quantity of the pH increaser to be added.

In order to achieve full virus inactivation, the adapter channel 214 can be formed between the two static mixers 278 such that the fluid remains at the reduced pH value for a sufficient time. This partial path is preferably meander-shaped such that the fluid remains at the reduced pH value preferably for 30 minutes.

We note that the arrangements of the adapter channel 214 shown in FIGS. 6a) to 6c) are exemplary representations. The adapter channel 214 can be arranged according to any of the embodiments described above. FIGS. 6a) to 6c) in particular describe an arrangement wherein all steps or all components of a virus inactivation are specified in one adapter plate 200. Several adapter plates 200 can also be arranged in series or can be coupled to each other, and the individual components can be distributed among these adapter plates 200. This means that the virus inactivation is accomplished when the fluid flow 14 has traveled through the individual adapter plates 200 with the various components for virus inactivation.

Auxiliary pumps can be arranged in the adapter plate 200, as shown in FIGS. 6b) and 6c). However, an auxiliary pump can also be arranged outside of an adapter plate 200, or as an external device.

As described for FIG. 6c), the partial path can also run between the two static mixers 278, and also at least partially outside of the adapter plate 200.

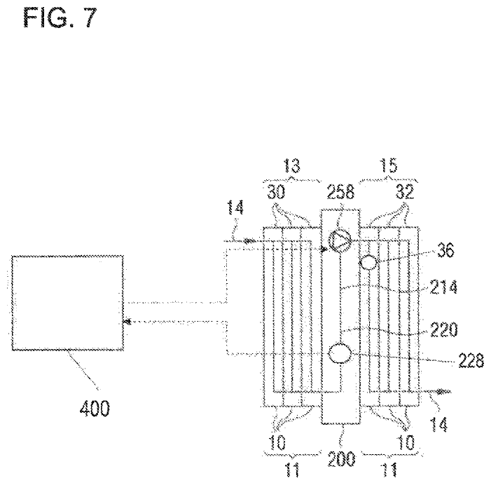

FIG. 7 shows a processing system 100 having a first and second processing unit group 13 and 15. The first and second processing unit group 13 and 15 are coupled by an adapter plate 200. At least one pump 258 is arranged in or on the adapter channel 214 of the adapter plate 200. The latter can be used when two processing unit groups 11 are arranged in parallel, but is particularly advantageous in an in-series arrangement, as shown in FIG. 7, because this permits controlling the pressure that the fluid exerts on the processing unit 10 located downstream in flow direction. In particular a flow resistance can be overcome by these means such that a sufficient filtration performance can be achieved in the second processing unit group 15.

The pump 258 can also in particular be formed as a displacement pump in order to initiate a vacuum in the first processing unit group 13 and to build up a desired filtration pressure in the second processing unit group 15. The pump 258 can in particular be formed as a cost-effective single-use component.

The pump 258 is coupled to the external control device 400. The pump 258 receives control commands from the external control device 400 that can activate, deactivate, or control or regulate the pump 258.

The pump 258 is preferably controlled or regulated based on measurement data of at least one adapter plate sensor 228 and/or at least one processing unit sensor 36 that have recorded the at least one parameter of the fluid flow 14 in the processing system 100. Measurement data of the at least one sensor can for example be read out and used by the external control device to regulate the pump 258 in order to achieve the desired pressure of the fluid flow 14. Limit values can for this purpose be specified in the external control device 400. If the pressure of the fluid flow 14 deviates from the defined values, the external control device 400 can adjust the settings of the pump 258 such that the fluid flow 14 has the desired pressure. The settings on the pump 258 can be adjusted by a pump actuator that receives the control commands from the external control device 400.

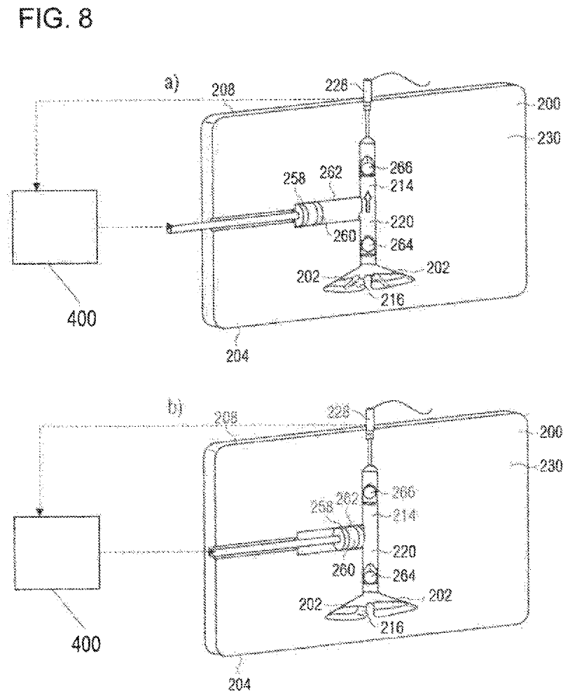

FIGS. 8a) and 8b) show a concrete embodiment of an adapter plate 200 having a displacement pump, specifically a piston pump. The pump 258 is preferably arranged in the connecting channel area 220 and comprises at least one piston 260 that is designed to execute a stroke motion, that is to say a linear (translational) motion. The piston 260 is for this purpose arranged in a cylinder 262. The pump 258 additionally has an inlet and an outlet that can each be sealed by a valve.

Specifically, a section in the connecting channel area 220 is split off or segregated in flow direction by an inlet valve 264 and by an outlet valve 266. Using the inlet valve 264 the fluid can flow into this section, and can flow out of this section using the outlet valve 266. The cylinder 262 that accommodates the piston 260, and in which the piston 260 can perform a translational motion, is arranged such that the cylinder 262 is arranged on the connecting channel area 220 and is fluidically connected to the latter. The cylinder 262 is in particular arranged between the inlet and outlet valve 264 and 266 on the connecting channel area 220.

During a first stroke, the intake, the piston 260 performs a reverse motion, that is to say a motion away from the connecting channel area 220. The inlet valve 264 opens and the fluid to be conveyed can flow into the connecting channel area 220 or the cylinder 262. During a second stroke, the conveyance motion, the inlet valve 264 closes, and the piston 260 moves in the direction toward the connecting channel area 220. The outlet valve 266 opens and the conveyed medium is pushed out.

At least one adapter plate sensor 228 is preferably arranged in the adaptive channel 214 to monitor the fluid flow 14. For this purpose, the adapter plate sensor 228 can preferably be arranged in flow direction downstream of the pump 258, for example to measure the fluid pressure or the fluid flow. This value can be used to regulate the pump 258 using the external control device 400.

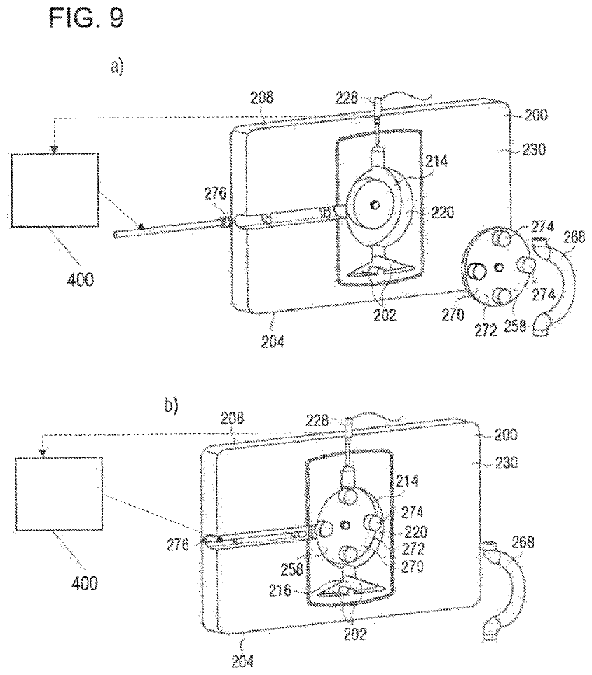

A further option of a displacement pump is shown based on FIGS. 9a) and 9b). The pump 258 integrated into the adapter plate 200 is a peristaltic pump. The latter is for the reasons already described above preferably arranged in the connecting channel area 220.

A peristaltic pump, also called a hose pump, is a displacement pump wherein the fluid to be conveyed is pushed through a hose 268 by an external mechanical deformation of the latter. In the present case, the hose 268 therefore represents a part of the connecting channel area 220 through which the fluid flows into the adapter plate 200. The area in which the hose 268 is arranged is circular, that is to say the connecting channel area 220 is expanding in an arc shape. A rotor 270 is arranged in the circular section of the connecting channel area 220, and is rotatably arranged therein.

The rotor 270 is preferably formed as a round, circular plate. At least one roller 274 and/or one sliding block is/are arranged on an upper side 272 of the rotor 270. The hose 268 is located at least sectionally on a jacket surface of the circular section of the connecting channel area 220. The hose 268 can be pinched off from the interior by the rollers 274 and/or the sliding blocks based on a rotation of the rotor 270. This has the effect that a pinched off location travels along the hose 268, thus moving the fluid to be conveyed forward. As shown in FIGS. 9a) and 9b), the rotor 270 is rotatable by a gear mechanism 276. At least one adapter plate sensor 228 can be arranged in the adapter plate 200, as was described in the embodiments above. The gear mechanism 276 can in particular be controlled by the external control device 400, preferably using an actuator. The control commands from the external control device 400 are in this case at least partially based on measurement values of the at least one adapter plate sensor 228 and/or the at least one processing unit sensor 36 in the processing system 100.

Further annotations regarding the embodiments described above:

The adapter plate 200 to link to processing unit groups 11 is described in various embodiments. However, we note that the descriptions apply likewise for embodiments that specify only a first and/or second processing unit 30 and 32.

The described processing systems 100 each show one adapter plate 200 that couples to processing unit groups 11. It is however possible that a processing system 100 contains a plurality of adapter plates 200. Processing unit groups 11 that are coupled by an adapter plate 200 are herein always designated as a first and second processing unit group 13 and 15, and the above description applies accordingly to any link between two processing unit groups 11. The same also applies to processing units 10 linked by an adapter plate 200, or to individual processing units 10 linked to a processing unit group 11.

The embodiments described above describe a pump 258 in the adapter plate 200 as a means for regulating the pressure of the fluid flow 14. Alternatively or additionally to the pump 258, at least one valve can be arranged in the adapter channel 214. The valve is designed to constrict the adapter channel 214 or to vary the size of the cross-section of the adapter channel 214, so as to regulate the pressure of the fluid flow 14. The valve can be controlled by the external control device 400. The control signals are in particular based on measured pressure parameters that were measured by the at least one adapter plate sensor 228 and/or the processing unit sensor 36.

The embodiments described above of pumps 258 in an adapter plate 200 can alternatively be formed as a compressed air pump, wherein preferably at least one membrane is moved up and down such that the fluid is moved in flow direction.

The valves described above can principally be actuated electrically, mechanically, pneumatically, or hydraulically.

We also note that although the shown adapter plates 200 are formed as two parts, it is also conceivable that these are formed as a single part.

The description above addresses the biopharmaceutical use of the processing systems 100 or the adapter plates 200. However, it is also possible to apply the shown principle to other processes, such as food processing, chemical production processes, beverage filtration, particle fractionation, wastewater treatment, etc.

In particular, we note that in the described embodiments, the adapter plate 200 is in each case coupled to a second processing unit 32 directly, that is to say without arranging further elements in-between. It is however possible to connect the adapter plate 200 to the second processing unit 32 using a compatible connector. The latter is preferably formed sterile and/or drip-free. Tanks or other components can also be arranged in-between.

The adapter plate 200 can also be arranged upstream and/or downstream of the last processing unit 10 of a processing system 100. In particular a filter cassette “Sartoclear® Depth Filter Cassette” and/or a filter cassette of the “Sartoclear® DL Series” and/or a filter cassette of the “Sartoclear® S Series” from Sartorius Stedim Biotech GmbH can be used as one or several processing units 10.

The various embodiments of the processing systems 100 or the adapter plates 200 were described separately based on the individual figures. However, we note that the individual embodiments or parts of the individual embodiments can be combined with each other. In an effort to avoid repetitions, elements already described for individual embodiments where also not described yet again.

The following now describes the signal transmission in the processing system 100:

As already discussed in detail above, the at least one adapter plate 200 and/or the at least one processing unit 10 each has/have at least one sensor. The latter measures at least one parameter of the fluid flow 14 in the processing system 100. The at least one sensor is coupled to the external control device 400 either by cable or wirelessly such that measurement values of the at least one sensor can be transmitted to the external control device 400.

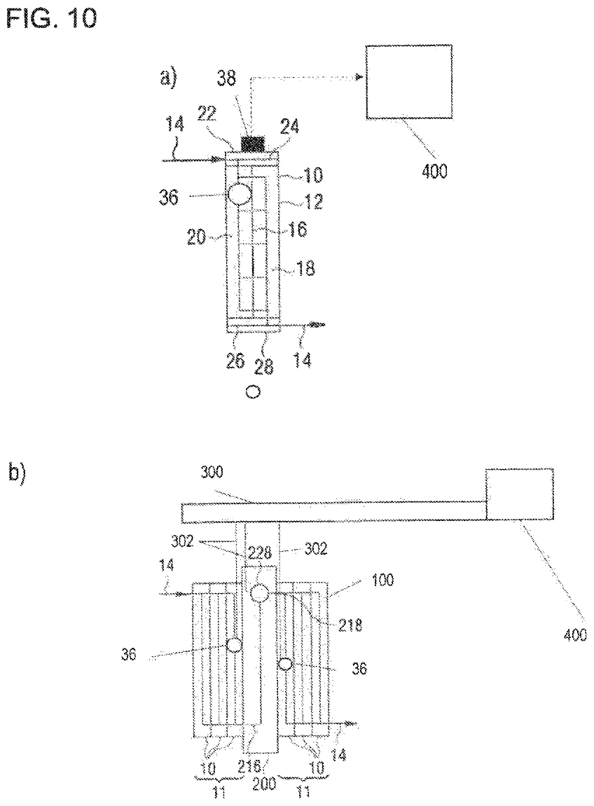

FIG. 10a) shows a processing unit 10 in which a processing unit sensor 36 is specified or embedded.

The latter, for example, measures the pressure of the fluid flow 14 before the fluid contacts the filter medium 16. As a result, it is possible to monitor whether the fluid flow 14 has a sufficient pressure such that a satisfactory filter performance can be achieved.

The processing unit 10 has at least one processing unit transponder 38 that is arranged in or on the processing unit 10. FIG. 10a) shows an example of a processing unit transponder 38 that is arranged on the processing unit 10. The processing unit transponder 38 is coupled to the processing unit sensor 36 either by cable, wirelessly, or optically, and receives measurement data from the processing unit sensor 36. The measurement data are in particular transmitted together with a unique address of the processing unit sensor 36 such that the measurement data can be unambiguously mapped to a sensor. These data are transmitted from the processing unit transponder 38 by radio to the external control device 400. The transmission is preferably performed automatically.

The processing unit transponder 36 can be formed as a passive transponder, preferably as a “Radio Frequency Identification Device” (RFID), or as an active transponder with a dedicated power supply. For this purpose, the processing unit transponder 36 has a rechargeable battery or is connected to an external power main. The processing unit transponder 36 can in particular use the “Near Field Communication” (NFC) data transmission standard.

Data can preferably be transmitted by WiFi, Bluetooth, and/or mobile radio standard (such as LTE, 5G, GSM).

If the processing unit 10 has several processing unit sensors 36, all or at least several processing unit sensors 36 can transmit their measurement data to the external control device 400 using one processing unit transponder 38. Alternatively, the processing unit sensor 36 can be equipped with an integrated transponder such that each processing unit sensor 36 has its own transponder.

The use of transponders for wireless data transmission of sensor data to the external control device 400 was discussed on an exemplary basis based on a processing unit 10. If the processing system 100 has several processing units 10 that each have at least one processing unit sensor 36, the latter or at least parts thereof can jointly use one processing unit transponder 38 for data transmission. The jointly used processing unit transponder 38 can be arranged in or on one of the processing units 10, or can be formed as an external unit.

We note that use of at least one transponder for wireless data transmission was described here on an exemplary basis for at least one processing unit. However, at least one transponder can be also used accordingly for at least one adapter plate sensor 228 in at least one adapter plate 200. Alternatively, there is also the option that at least one processing unit sensor 36 and at least one adapter plate sensor 228 jointly use one transponder. The latter can be arranged in or on a processing unit 10, on an adapter plate 200, or as an external device.

FIG. 10b) shows an embodiment of a processing system 100 having a cable-based data transmission from the at least one sensor to the external control device 400.

FIG. 10b) specifically shows a processing system 100 with two processing unit groups 11 that are coupled by an adapter plate 200. On an exemplary basis, two of the processing units 10 have a processing unit sensor 36, and the adapter plate 200 has an adapter plate sensor 228. As was already described above, the number of processing units 10 and the number of adapter plates 200 are however both variable. Furthermore, the number and the locations of sensors in the processing system 100 are variable based on the descriptions above for other embodiments.

The individual sensors 228, 36 are in this case coupled to the external control device 400 using a bus system 300. The bus system 300 comprises at least one bus conductor that is preferably routed along the at least one processing unit 10 and the at least one adapter plate 200. The individual sensors 228, 36 of the processing system 100 are coupled to the at least one bus conductor using the cable 302. Every sensor 228, 36 can for this purpose be coupled to a separate bus conductor, or several sensors 228, 36 can use the same bus conductor. In particular, the cables 302 of several sensors 228, 36 that are embedded into a processing unit 10 or into an adapter plate 200 can initially be merged in the processing unit 10 or in the adapter plate 200, such that only one cable 302 is routed out of the processing unit 10 or the adapter plate 200, and is then coupled to a bus conductor. The measurement data of sensors 228, 36 are transmitted according to the wireless data transmission as described for FIG. 10a), and are analyzed by the external control device 400.

The bus conductors can preferably be arranged in at least one rail that is routed along the at least one processing unit 10 and the at least one adapter plate 200.

The sensors 228, 36 can be equipped with a rechargeable battery that supplies the sensors 228, 36 with the required power during their operating time. Alternatively, the individual sensors 228, 36 can each be supplied with power using an external power source. The at least one cable that is required to supply the sensors 228, 36 with power can for this purpose also be routed in the rail described above and subsequently be coupled to the individual sensors 228, 36. If several sensors 228, 36 are located in a processing unit 10 or an adapter plate 200, it is preferred that respectively one primary power supply cable protrudes from the processing unit 10 and the adapter plate 200 that can then be coupled to a primary cable that for example is routed in the rail of the bus system 300. The primary power supply cable is connected to the individual sensors 228, 36 in the processing unit 10 or the adapter plate 200. The user can then in only a few steps supply the sensors 228, 36 with power.

Alternatively, the at least one processing unit 10 and the at least one adapter plate 200 have an integrated power supply. For this purpose, the at least one processing unit 10 and the at least one adapter plate 200 are each integrated into at least a subsection of a power supply conductor. The subsections of the power supply conductor can be connected to each other as soon as the individual components (processing units, adapter plates) are joined. The power supply conductor is fed at a single point by an external power source. The individual sensors 228, 38 are connected to the power supply conductor. One of the termination brackets 34 that hold the processing units 10 and the adapter plates 200 in the manner of a sandwich preferably also comprises a subsection of the power supply conductor, and the external power source is connected to the subsection of the power supply conductor in the termination bracket 34.

The power supply was described above strictly on an exemplary basis for sensors 228, 36 of the processing system 100. However, we note that all other elements of the processing system 100 that require power also either have a rechargeable battery, or can be fed using any of the cable-based power supply options described above. For this purpose, reference is for example made to the actuators of the deflection elements, the pump 258, or to the at least one valve for controlling pressure in the adapter plate 200.

The bus system 300 can also be used for this purpose in order to transmit control commands from the external control device 400 to the individual components, such as the actuators or the deflection elements, the at least one valve for pressure control, or the pump 258 in the adapter plate 200. For this purpose at least one control bus conductor is specified in the bus system 300 that permits transmitting data from the external control device 400 to the element to be controlled. Any of the elements to be controlled can for this purpose be directly linked to a separate or common control bus conductor. Alternatively, a primary data conductor can for example be routed from the corresponding control bus conductor into the adapter plate 200, wherein the primary data conductor is split in the adapter plate 200 to connect the individual components to be controlled.

Alternatively to a cable-based data transmission, the control signals can also be transmitted wirelessly, as was already described regarding the data transmission between the sensors 228, 36 and in the external control device 400. The external control device 400 can for this purpose have its own transponder that transmits control commands to the corresponding components in the processing system 100, which in turn facilitate regulating the fluid flow 14.

The processing system 100 according to the invention therefore provides a plurality of advantages. Firstly, at least one sensor 228, 36 is embedded in the processing system 100 such that elaborate connecting, calibrating, and sterilizing of the sensor 228, 36 is omitted. Moreover, the processing system 100 only comprises one external control device 400 by which the measurement data of the sensors 228, 36 are collected and simultaneously used to regulate the fluid flow 14. For the user, there is then only one point of contact by which he can monitor and at the same time regulate the processing system 100.

Moreover, the data transmission is provided to the user in a simple manner. Both, a wireless and also a cable-based data transmission between the external control device 400 and the individual components of the processing system 100 that communicate with the external control device 400 can be provided in a straightforward and fast manner. Elaborate cabling is avoided. A compact and user-friendly processing system 100 is provided as a result.

### LIST OF REFERENCE SYMBOLS

10 Processing unit

11 Processing unit group

12 Processing housing

13 First processing unit group

14 Fluid flow

15 Second processing unit group

16 Filter medium

17 Filter carrier

18 Filtrate side

19 Vacant space

20 Retentate side

21 Bulk medium

22 Upper end of the processing housing

24 Inlet channel

26 Outlet channel

27 Second outlet channel

30 First processing unit

32 Second processing unit

34 Termination bracket

35 Inlet pump

36 Processing unit sensor

38 Processing unit transponder

40 Filter cartridge

42 First side of the filter cartridge

44 Second side of the filter cartridge

46 Filter cartridge valve

100 Processing system

200 Adapter plate

202 First inlet opening

204 Bottom end of the adapter plate

206 Second inlet opening

208 Upper end of the adapter plate

210 First outlet opening

212 Second outlet opening

214 Adapter channel

216 First channel area

218 Second channel area

220 Connecting channel area

222 Deflection valve

224 First deflection valve

226 Second deflection valve

228 Adapter plate sensor

230 Inlet plate

234 Channel depression

236 Flow web

238 Connecting channel

240 Auxiliary branch

242 Channel element

244 Auxiliary inlet or outlet

246 Multiway valve

248 Valve tube

250 Jacket surface of the valve tube

252 Valve opening

254 Actuation arm

257 Deflection actuator

258 Pump

260 Piston

262 Cylinder

264 Inlet valve

266 Outlet valve

268 Hose

270 Rotor

272 Upper side of the rotor

274 Roller

276 Gear mechanism

278 Static mixer

300 Bus system

302 Cable

400 External control device

HR Horizontal direction

VR Vertical direction

1. A modular processing system for biopharmaceutical and/or chemical processes, comprising:
at least one processing unit;
at least one adapter plate that can be directly or indirectly fluidically connected to the processing unit, wherein the adapter plate has at least one adapter channel through which at least one fluid flow can flow, which flows to the processing unit, wherein the adapter plate additionally has at least one deflection element and/or a pump and/or at least one valve; and
an external control device;
wherein the adapter plate is designed in such a way that the fluid flow to the processing unit can be at least partially deflected with the at least one deflection element in the adapter channel and/or the fluid flow, preferably its pressure, is controllable with the at least one valve and/or the pump in the adapter channel;
wherein at least one sensor is embedded in the processing unit and/or in the adapter plate, in order to detect at least one property of the fluid flow in the processing unit or the adapter plate; and
wherein the external control device is coupled to the at least one sensor in such a way that measurement data of at least one sensor can be read out, and the fluid flow in the processing unit and or the adapter plate can be centrally controlled based on the read-out measurement data.

2. The modular processing system according to claim 1, comprising at least a first and a second processing unit that can be fluidically connected to each other,
wherein at least one fluid flow that flows from the first processing unit to the second processing unit can flow through the at least one adapter channel of the adapter plate; and
wherein the adapter plate is designed in such a way that the fluid flow between the first processing unit and the second processing unit can be at least partially deflected with the at least one deflection element in the adapter channel and the fluid flow, preferably its pressure, is controllable with the at least one valve and/or the pump in the adapter channel.

3. The modular processing system according to claim 1, wherein respectively at least one sensor is embedded in the first and second processing unit and in the adapter plate, in order to detect at least one property of the fluid flow in the first and second processing unit and the adapter plate.

4. The modular processing system according to claim 3, wherein the external control device is coupled to the sensors in such a way that measurement data of the sensors can be read out, and the fluid flow in the processing units and or the adapter plate can be centrally controlled based on the read-out measurement data.

5. The modular processing system according to claim 1, wherein the at least one processing unit and/or the adapter plate each have at least one transponder that is designed to transmit measurement data of a corresponding sensor to the external control device; or
wherein the sensors of the at least one processing unit and/or the at least one adapter plate are coupled to the external control device using a bus system.

6. The modular processing system according to claim 1, wherein the at least one deflection element and/or the pump and/or the at least one valve are controllable by an actuator.

7. The modular processing system according to claim 1, wherein the sensor is formed to measure a pressure, a volumetric flow, a UV value, a pH value, a turbidity, and or a viscosity of the fluid flow.

8. The modular processing system according to claim 1, wherein the at least one sensor, the at least one deflection element, the at least one valve, and/or the pump, each have a rechargeable battery.

9. The modular processing system according to claim 1, wherein the at least one sensor, the at least one deflection element, the at least one valve, and/or the pump have a cable-based power supply.

10. The modular processing system according to claim 9, wherein the processing system has a central power supply for the at least one sensor, the at least one deflection element, the at least one valve and/or the pump,
wherein the at least one processing unit and the at least one adapter plate have partial sections of the power supply that form the central power supply when assembled.

11. The modular processing system according to claim 1, wherein the at least one sensor, the at least one deflection element, the at least one valve, and the pump are formed as single-use elements.

12. A method for centrally controlling a modular processing system for biopharmaceutical and/or chemical processes, comprising the steps:
Provide at least one processing unit;
Provide at least one adapter plate that can have at least one adapter channel through which at least one fluid flow can flow, wherein the adapter plate additionally has at least one deflection element and/or a pump and/or at least one valve;
Provide an external control device;
Directly or indirectly connect the adapter plate to the processing unit such that the fluid flow can flow from the adapter plate to the processing unit;
Detect at least one property of the fluid flow in the processing unit and/or the adapter plate using at least one sensor that is embedded in the processing unit and/or the adapter plate; and
Couple the external control device to the at least one sensor such that measurement data from the at least one sensor can be read out; and
Couple the external control device to the at least one deflection element and/or the pump and/or the at least one valve in the adapter plate such that the fluid flow in the processing unit and/or the adapter plate can be centrally controlled based on the read-out measurement data;
wherein the fluid flow is at least partially deflected using the at least one deflection element in the adapter channel, and/or
wherein the fluid flow, preferably its pressure, can be regulated using the at least one valve and/or the pump in the adapter channel.

13. The method according to claim 12, wherein the processing system comprises at least one first and second processing unit; and
wherein the first and second processing unit are coupled to each other by the adapter plate such that the fluid flow can flow from the first processing unit to the second processing unit.

14. The method according to claim 13, wherein respectively at least one sensor is embedded in the first and second processing unit and in the adapter plate;
wherein the sensors detect at least one property of the fluid flow in the first and second processing unit and the adapter plate; and
wherein the sensors are coupled to the external control device such that measurement data of the sensors can be read out, and the fluid flow in the processing units and or the adapter plate can be centrally controlled based on the read-out measurement data.
